SEQUENCE LISTING (26 sequences)

SEQ ID NO: 1 - protein, 522 aa, Artificial Sequence
  microRNA binding protein
IEIKVWAIACFAPQRQCTEVHLKSFTEQLRKISRDAGMPIQGQPCFCKYAQGADSVEPMFRHLKNTYAGLQLVVVILPGKTPVYAEVKRVGDTVLGMATQCVQMKNVQRTTPQTLSNLCLKINVKLGGVNNILLPQGRPPVFQQPVIFLGADVTHPPAGDGKKPSIAAVVGSMDAHPNRYCATVRVQQHRQEIIQDLAAMVRELLIQFYKSTRFKPTRIIFYRDGVSEGQFQQVLHHELLAIREACIKLEKDYQPGITFIVVQKRHHTRLFCTDKNERVGKSGNIPAGTTVDTKITHPTEFDFYLCSHAGIQGTSRPSHYHVLWDDNRFSSDELQILTYQLCHTYVRCTRSVSIPAPAYYAHLVAFRARYHLVDKEHDSAEGSHTSGQSNGRDHQALAKAVQVHQDTLRTMYFASRLEDIGGGGGGEQAPEDQGPQREPHNEWTLELLEELKREAVRHFPRPWLHGLGQHIYETYGDTWAGVEAIIRILQQLLFIHFRIGCQHSRIGIIQQRRARRNGASRS

SEQ ID NO: 2 - protein, 282 aa, Artificial Sequence
  EPN-01
MGARASGSKSGSGSDSGSKIEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEQKLISEEDLQSRPEPTAPPEESFRSGVETTTPPQKQEPIDKELYPLTSLRSLFGNDPSSQ

SEQ ID NO: 3 - protein, 281 aa, Artificial Sequence
  EPN-03
MGAQFSGSKSGSGSDSGSKIEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEQKLISEEDLQSRPEPTAPPEESFRSGVETTTPPQKQEPIDKELYPLTSLRSLFGNDPSS

SEQ ID NO: 4 - protein, 288 aa, Artificial Sequence
  EPN-07
MGCIKSKRKDNLNGSKSGSGSDSGSKIEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEQKLISEEDLQSRPEPTAPPEESFRSGVETTTPPQKQEPIDKELYPLTSLRSLFGNDPSS

SEQ ID NO: 5 - protein, 282 aa, Artificial Sequence
  EPN-08
MGCTLSAEERAALGSKSGSGSDSGSKIEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEQKLISEEDLQSRPEPTAPPEESFRSGVETTTPPQKQEPIDKELYPLTSLRSLF

SEQ ID NO: 6 - protein, 399 aa, Artificial Sequence
  EPN-018
MHGLQDDPDLQALLKGSQLLKVKSSSWRRERFYKLQEDCKTIWQESRKVMRSPESQLFSIEDIQEVRMGHRTEGLEKFARDIPEDRCFSIVFKDQRNTLDLIAPSPADAQHWVQGLRKIIHHSGSMDQRQKGSGSKIEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEQKLISEEDLQSRPEPTAPPEESFRSGVETTTPPQKQEPIDKELYPLTSLRSLFGNDPSSQ

SEQ ID NO: 7 - protein, 858 aa, Homo sapiens
MYSGAGPALAPPAPPPPIQGYAFKPPPRPDFGTSGRTIKLQANFFEMDIPKIDIYHYELDIKPEKCPRRVNREIVEHMVQHFKTQIFGDRKPVFDGRKNLYTAMPLPIGRDKVELEVTLPGEGKDRIFKVSIKWVSCVSLQALHDALSGRLPSVPFETIQALDVVMRHLPSMRYTPVGRSFFTASEGCSNPLGGGREVWFGFHQSVRPSLWKMMLNIDVSATAFYKAQPVIEFVCEVLDFKSIEEQQKPLTDSQRVKFTKEIKGLKVEITHCGQMKRKYRVCNVTRRPASHQTFPLQQESGQTVECTVAQYFKDRHKLVLRYPHLPCLQVGQEQKHTYLPLEVCNIVAGQRCIKKLTDNQTSTMIRATARSAPDRQEEISKLMRSASFNTDPYVREFGIMVKDEMTDVTGRVLQPPSILYGGRNKAIATPVQGVWDMRNKQFHTGIEIKVWAIACFAPQRQCTEVHLKSFTEQLRKISRDAGMPIQGQPCFCKYAQGADSVEPMFRHLKNTYAGLQLVVVILPGKTPVYAEVKRVGDTVLGMATQCVQMKNVQRTTPQTLSNLCLKINVKLGGVNNILLPQGRPPVFQQPVIFLGADVTHPPAGDGKKPSIAAVVGSMDAHPNRYCATVRVQQHRQEIIQDLAAMVRELLIQFYKSTRFKPTRIIFYRDGVSEGQFQQVLHHELLAIREACIKLEKDYQPGITFIVVQKRHHTRLFCTDKNERVGKSGNIPAGTTVDTKITHPTEFDFYLCSHAGIQGTSRPSHYHVLWDDNRFSSDELQILTYQLCHTYVRCTRSVSIPAPAYYAHLVAFRARYHLVDKEHDSAEGSHTSGQSNGRDHQALAKAVQVHQDTLRTMYF

SEQ ID NO: 8 - protein, 97 aa, HIV-1
MEQAPEDQGPQREPHNEWTLELLEELKREAVRHFPRPWLHGLGQHIYETYGDTWAGVEAIIRILQQLLFIHFRIGCQHSRIGIIQQRRARRNGASRS

SEQ ID NO: 9 - DNA, 9804 bp, Artificial Sequence
  VB200705-1156knk
caactttgtatagaaaagttgaagctttctggctaactagagaacccatcgatggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagccaccatgggtgcgcgtgcgtctggttctaaatctggctccggctctgactctggtagcaaaatcgaagagctgttcaagaaacacaagatcgttgccgtgctgcgtgccaatagtgtggaagaagcgaaaaagaaagcgctggcggttttcctgggcggcgttcatctgattgaaattacctttaccgtgccggatgcggataccgtgattaaggaactgagctttctgaaggaaatgggcgcgattattggtgcgggcaccgtgaccagcgtggagcagtgccgtaaagcggtggaaagtggcgccgaattcattgtgagtccgcacctggacgaggaaattagccaattttgcaaggagaagggtgtgttctatatgccaggcgttatgaccccgaccgaactggtgaaagccatgaaactgggccataccatcttaaaactgtttccgggtgaggtggtgggtccgcagtttgttaaagcgatgaaaggtccgtttccgaatgtgaaatttgtgccaaccggcggtgttaatctggacaatgtgtgcgaatggttcaaagcgggcgtgctggccgtgggcgtgggcagcgcgttagtgaaaggcaccccggtggaagtggcggaaaaggccaaggcgttcgttgagaagattcgtggctgcaccgaacagaaactgattagcgaagaagatctgcagtctcgtccggaaccgaccgcaccgccggaagaatctttccgttctggtgttgaaaccaccacgccaccgcaaaaacaggagccgatcgacaaagaactgtacccgctgacctctctgcgttctctgttcggtaacgacccgtcttctcagtaacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattaattaagggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggcctggagacgccatccacgctgttttgacctccatagaagacaccgggaccgatccagcctccggtcgaccgatcctgagaacttcaggggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctctttcctacagctcctgggcaacgtgctggttgttgtgctgtctcatcattttggcaaagaattcctcgacggatccctcgaggaattctgacactgccaccatgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgcccaagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtaattttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcagcccattaattaacaagtttgtacaaaaaagcaggctcgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggacccagctttcttgtacaaagtggtctagagccaccatggaacagaaactgattagcgaagaagatctggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtctagactcgaggatatcggaggtggcggtggaggtgaacaggcaccggaagatcaggggcctcagcgtgaaccgcataatgaatggaccctggaactgctggaagaactgaaacgtgaagcggtgcgtcatttcccgcgtccgtggctgcatggcctgggccagcatatctatgaaacctatggcgatacctgggctggcgtggaagcgattattcgtattctgcagcagctgttgtttattcattttcggataggctgccaacatagccggattggcattattcagcagcgtcgtgcgcgtcgtaatggcgcgagccgtagctaacaactttattatacatagttgatggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagcggccgcggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctctcttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcggcgcgccgcggccgc

SEQ ID NO: 10 - DNA, 28 bp, Artificial Sequence
  EcoR1-Myc-EGFP_F
gcgaattcttaattaatctagacatgga

SEQ ID NO: 11 - DNA, 28 bp, Artificial Sequence
  EF1A-EcoR1_R
gcgaattcagcctgcttttttgtacaaa

SEQ ID NO: 12 - DNA, 62 bp, Artificial Sequence
  VLP-Flag-Ago2_F
ttaattaatctagacatggattacaaagacgatgacgataagatgtactcgggagccggccc

SEQ ID NO: 13 - DNA, 35 bp, Artificial Sequence
  VLP-Ago2_R
atcctcgagtctagaagcaaagtacatggtgcgca

SEQ ID NO: 14 - DNA, 41 bp, Artificial Sequence
  p-delta-EGFP_Flag_F
gtcagatccgctagcgccaccatggattacaaagacgatga

SEQ ID NO: 15 - DNA, 35 bp, Artificial Sequence
  pRP_p-delta-EGFP-C2_R
cagaattcgaagctttttgtacaagaaagctgggt

SEQ ID NO: 16 - DNA, 65 bp, Artificial Sequence
  pdEGFP_Flag-Ago2_Paz-PIWI_F
gtcagatccgctagcgccaccatggattacaaagacgatgacgatgcacagccagtaatcgagtt

SEQ ID NO: 17 - DNA, 65 bp, Artificial Sequence
  pdEGFP_Flag-Ago2_L2-PIWI_F
gtcagatccgctagcgccaccatggattacaaagacgatgacgatgcaggacaaagatgtattaa

SEQ ID NO: 18 - DNA, 35 bp, Artificial Sequence
  pdEGFP_Flag-Ago2-dN-PAZ_F
tccgatatcctcgagagcaaagtacatggtgcgca

SEQ ID NO: 19 - DNA, 80 bp, Artificial Sequence
  Ago2-Xba-Vpr_R
cgtacctgtcagcacgctgttgaccttagcccgtggcaacgactgtgaagcctccgacacgtactgagggcgattcccag

SEQ ID NO: 20 - DNA, 28 bp, Artificial Sequence
  kpn1-SV40_F
gcggtaccacccagctttcttgtacaaa

SEQ ID NO: 21 - DNA, 31 bp, Artificial Sequence
  EPN01-kpn1_R
gcggtaccttactgagaagacgggtcgttac

SEQ ID NO: 22 - DNA, 35 bp, Artificial Sequence
  p-delta-EGFP_EPN01_F
gtcagatccgctagcgccaccatgggtgcgcgtgc

SEQ ID NO: 23 - protein, 976 aa, Artificial Sequence
  Flag-Ago2-FL-Vpr
MDYKDDDDKMYSGAGPALAPPAPPPPIQGYAFKPPPRPDFGTSGRTIKLQANFFEMDIPKIDIYHYELDIKPEKCPRRVNREIVEHMVQHFKTQIFGDRKPVFDGRKNLYTAMPLPIGRDKVELEVTLPGEGKDRIFKVSIKWVSCVSLQALHDALSGRLPSVPFETIQALDVVMRHLPSMRYTPVGRSFFTASEGCSNPLGGGREVWFGFHQSVRPSLWKMMLNIDVSATAFYKAQPVIEFVCEVLDFKSIEEQQKPLTDSQRVKFTKEIKGLKVEITHCGQMKRKYRVCNVTRRPASHQTFPLQQESGQTVECTVAQYFKDRHKLVLRYPHLPCLQVGQEQKHTYLPLEVCNIVAGQRCIKKLTDNQTSTMIRATARSAPDRQEEISKLMRSASFNTDPYVREFGIMVKDEMTDVTGRVLQPPSILYGGRNKAIATPVQGVWDMRNKQFHTGIEIKVWAIACFAPQRQCTEVHLKSFTEQLRKISRDAGMPIQGQPCFCKYAQGADSVEPMFRHLKNTYAGLQLVVVILPGKTPVYAEVKRVGDTVLGMATQCVQMKNVQRTTPQTLSNLCLKINVKLGGVNNILLPQGRPPVFQQPVIFLGADVTHPPAGDGKKPSIAAVVGSMDAHPNRYCATVRVQQHRQEIIQDLAAMVRELLIQFYKSTRFKPTRIIFYRDGVSEGQFQQVLHHELLAIREACIKLEKDYQPGITFIVVQKRHHTRLFCTDKNERVGKSGNIPAGTTVDTKITHPTEFDFYLCSHAGIQGTSRPSHYHVLWDDNRFSSDELQILTYQLCHTYVRCTRSVSIPAPAYYAHLVAFRARYHLVDKEHDSAEGSHTSGQSNGRDHQALAKAVQVHQDTLRTMYFASRLEDIGGGGGGEQAPEDQGPQREPHNEWTLELLEELKREAVRHFPRPWLHGLGQHIYETYGDTWAGVEAIIRILQQLLFIHFRIGCQHSRIGIIQQRRARRNGASRS

SEQ ID NO: 24 - protein, 750 aa, Artificial Sequence
  Flag-PAZ-PIWI-Vpr
MDYKDDDDKAQPVIEFVCEVLDFKSIEEQQKPLTDSQRVKFTKEIKGLKVEITHCGQMKRKYRVCNVTRRPASHQTFPLQQESGQTVECTVAQYFKDRHKLVLRYPHLPCLQVGQEQKHTYLPLEVCNIVAGQRCIKKLTDNQTSTMIRATARSAPDRQEEISKLMRSASFNTDPYVREFGIMVKDEMTDVTGRVLQPPSILYGGRNKAIATPVQGVWDMRNKQFHTGIEIKVWAIACFAPQRQCTEVHLKSFTEQLRKISRDAGMPIQGQPCFCKYAQGADSVEPMFRHLKNTYAGLQLVVVILPGKTPVYAEVKRVGDTVLGMATQCVQMKNVQRTTPQTLSNLCLKINVKLGGVNNILLPQGRPPVFQQPVIFLGADVTHPPAGDGKKPSIAAVVGSMDAHPNRYCATVRVQQHRQEIIQDLAAMVRELLIQFYKSTRFKPTRIIFYRDGVSEGQFQQVLHHELLAIREACIKLEKDYQPGITFIVVQKRHHTRLFCTDKNERVGKSGNIPAGTTVDTKITHPTEFDFYLCSHAGIQGTSRPSHYHVLWDDNRFSSDELQILTYQLCHTYVRCTRSVSIPAPAYYAHLVAFRARYHLVDKEHDSAEGSHTSGQSNGRDHQALAKAVQVHQDTLRTMYFASRLEDIGGGGGGEQAPEDQGPQREPHNEWTLELLEELKREAVRHFPRPWLHGLGQHIYETYGDTWAGVEAIIRILQQLLFIHFRIGCQHSRIGIIQQRRARRNGASRS

SEQ ID NO: 25 - protein, 629 aa, Artificial Sequence
  Flag-L2-PIWI-Vpr
MDYKDDDDKAGQRCIKKLTDNQTSTMIRATARSAPDRQEEISKLMRSASFNTDPYVREFGIMVKDEMTDVTGRVLQPPSILYGGRNKAIATPVQGVWDMRNKQFHTGIEIKVWAIACFAPQRQCTEVHLKSFTEQLRKISRDAGMPIQGQPCFCKYAQGADSVEPMFRHLKNTYAGLQLVVVILPGKTPVYAEVKRVGDTVLGMATQCVQMKNVQRTTPQTLSNLCLKINVKLGGVNNILLPQGRPPVFQQPVIFLGADVTHPPAGDGKKPSIAAVVGSMDAHPNRYCATVRVQQHRQEIIQDLAAMVRELLIQFYKSTRFKPTRIIFYRDGVSEGQFQQVLHHELLAIREACIKLEKDYQPGITFIVVQKRHHTRLFCTDKNERVGKSGNIPAGTTVDTKITHPTEFDFYLCSHAGIQGTSRPSHYHVLWDDNRFSSDELQILTYQLCHTYVRCTRSVSIPAPAYYAHLVAFRARYHLVDKEHDSAEGSHTSGQSNGRDHQALAKAVQVHQDTLRTMYFASRLEDIGGGGGGEQAPEDQGPQREPHNEWTLELLEELKREAVRHFPRPWLHGLGQHIYETYGDTWAGVEAIIRILQQLLFIHFRIGCQHSRIGIIQQRRARRNGASRS

SEQ ID NO: 26 - protein, 531 aa, Artificial Sequence
  Flag-MID-PIWI-Vpr
MDYKDDDDKIEIKVWAIACFAPQRQCTEVHLKSFTEQLRKISRDAGMPIQGQPCFCKYAQGADSVEPMFRHLKNTYAGLQLVVVILPGKTPVYAEVKRVGDTVLGMATQCVQMKNVQRTTPQTLSNLCLKINVKLGGVNNILLPQGRPPVFQQPVIFLGADVTHPPAGDGKKPSIAAVVGSMDAHPNRYCATVRVQQHRQEIIQDLAAMVRELLIQFYKSTRFKPTRIIFYRDGVSEGQFQQVLHHELLAIREACIKLEKDYQPGITFIVVQKRHHTRLFCTDKNERVGKSGNIPAGTTVDTKITHPTEFDFYLCSHAGIQGTSRPSHYHVLWDDNRFSSDELQILTYQLCHTYVRCTRSVSIPAPAYYAHLVAFRARYHLVDKEHDSAEGSHTSGQSNGRDHQALAKAVQVHQDTLRTMYFASRLEDIGGGGGGEQAPEDQGPQREPHNEWTLELLEELKREAVRHFPRPWLHGLGQHIYETYGDTWAGVEAIIRILQQLLFIHFRIGCQHSRIGIIQQRRARRNGASRS

(19) United States
(12) Patent Application Publication
(10) Pub. No.: US 2024/0318163 A1
(43) Pub. Date: Sep. 26, 2024

(54) METHOD FOR NONINVASIVELY ACQUIRING MICRO-RNA FROM CELLS

(71) Applicant: NATIONAL INSTITUTE OF ADVANCED INDUSTRIAL SCIENCE AND TECHNOLOGY, Chiyoda-ku, Tokyo (JP)

(72) Inventors: Fumio Maeda, Koto-ku, Tokyo (JP); Shungo Adachi, Koto-ku, Tokyo (JP); Tohru Natsume, Koto-ku, Tokyo (JP)

(21) Appl. No.: 18/576,490

(22) PCT Filed: Jun. 15, 2022
(86) PCT No.: PCT/JP2022/023981

§ 371 Date: Jan. 4, 2024

(30) Foreign Application Priority Data
Jul. 8, 2021 (JP) 2021-113644

(51) Int. Cl. C12N 15/10

(52) U.S. Cl. CPC C12N 15/1006; C12N 15/1017

(57) ABSTRACT

A method for noninvasively acquiring a microRNA from a cell, the method comprising (1) introducing a nucleic acid coding a microRNA-binding protein and a nucleic acid coding a vesicle-forming protein into the cell, wherein the microRNA-binding protein comprises a first portion consisting of an MID domain and a PIWI domain of an Argonaute protein and a second portion consisting of a virus protein R, and the vesicle-forming protein comprises: a palmitoylation or myristoylation signal or a PH domain; a self-assembling domain; an ESCRT or ESCRT-related factor-binding domain; and a Gag p6 domain; thereby producing an exosome-like vesicle comprising a micro-RNA, (2) collecting an extracellular fluid of the cell, and (3) extracting the micro-RNA from the extracellular fluid.

### TECHNICAL FIELD

The present invention relates to a method for noninvasively acquiring microRNA from a cell.

### BACKGROUND ART

MicroRNAs (miRNAs) are small, single-stranded non-coding RNAs having a length of about 22 bases, and interact with mRNAs to control expression of the mRNAs. It has been revealed that miRNAs are involved in various biological processes such as cell proliferation, differentiation, apoptosis, and the onset and pathology of various diseases including cancer, and it has been reported that miRNAs have the potential to serve as biomarkers that reflect the state of cells (Non-Patent Document 1). The miRNAs are encapsulated in exosomes secreted from cells and are present in body fluid such as blood or urine, and thus liquid biopsy, which noninvasively diagnoses a disease by detecting and quantifying miRNA in body fluid, is attracting attention. In addition, miRNAs are closely related to the onset and pathology of various diseases, and thus, miRNA-containing exosomes are also anticipated as oligonucleotide therapeutics.

However, miRNA are contained in exosomes only at the concentration in cytoplasm because exosomes are formed by incorporating the cytoplasm. Thus, the amount of miRNAs contained in exosomes is extremely small, and it is difficult to obtain a sufficient amount of miRNAs for accurate diagnosis and drug development.

In recent years, a method for inducing the production of exosome-like extracellular vesicles by using an artificially designed self-assembling protein nanocage has been proposed (Patent Document 1, Non-Patent Document 2). According to this method, exosome-like vesicles that contain a target recombinant protein expressed in the cell can be obtained. However, as well as exosomes, the extracellular vesicles produced this method are also formed by incorporating the cytoplasm, and thus, it is still difficult to acquire miRNAs that are intrinsically present in low abundance in the cytoplasm.

### CITATION LIST

### Patent Document

[Patent Document 1] WO 2016/138525

### Non-Patent Document

[Non-Patent Document 1] Liang, Y. et al., Journal of Extracellular Vesicles, (2019), 9(1):1697583, DOI: 10.1080/20013078.2019.1697583
[Non-Patent Document 2] Votteler, J. et al., Nature, (2016), 540(7632): 292-295, DOI: 10.1038/nature20607

### SUMMARY OF INVENTION

### Technical Problem

The present invention has been made for the purpose of providing a method for noninvasively acquiring more miRNAs from cells.

### Solution to Problem

The inventors have succeeded in increasing the yield of miRNAs by de novo designing a miRNA binding protein having a size at which the protein can be encapsulated in an exosome-like vesicle.

That is, according to one embodiment, the present invention provides a method for noninvasively acquiring a microRNA from a cell, the method comprising: (1) introducing a nucleic acid coding a microRNA-binding protein and a nucleic acid coding a vesicle-forming protein into the cell, wherein the microRNA-binding protein comprises a first portion consisting of an MID domain and a PIWI domain of an Argonaute protein and a second portion consisting of a virus protein R, and wherein the vesicle-forming protein comprises: a palmitoylation or myristoylation signal or a pleckstrin homology domain; a self-assembling domain; an ESCRT or ESCRT-related factor-binding domain; and a Gag p6 domain; thereby an exosome-like vesicle including a microRNA is produced, (2) collecting an extracellular fluid of the cell, and (3) extracting the microRNA from the extracellular fluid.

The microRNA-binding protein preferably comprises an amino acid sequence having at least 90% identity to the amino acid sequence of SEQ ID NO: 1.

The vesicle-forming protein preferably comprises an amino acid sequence having at least 90% identity to an amino acid sequence selected from the group consisting of SEQ ID NOs: 2 to 6.

The cell may be an in vitro cell, and the extracellular fluid may be a culture supernatant.

The cell may be an in vivo cell, and the extracellular fluid may be a biological fluid.

In addition, according to one embodiment, the present invention provides an exosome-like vesicle comprising (a) a microRNA, (b) a microRNA-binding protein comprising a first portion consisting of an MID domain and a PIWI domain of an Argonaute protein and a second portion consisting of a virus protein R, and (c) a nanocage composed of a vesicle-forming protein that comprises an amino acid sequence having at least 90% identity to the amino acid sequence selected from the group consisting of SEQ ID NOs: 2 to 6.

The exosome-like vesicle preferably further comprises (d) a membrane fusion protein.

### Advantageous Effects of Invention

According to the method of the present invention, an extracellular fluid that contains miRNAs at a high concentration can be acquired. Thus, the method of the present invention enables noninvasive miRNA analysis, as is conventional miRNA analysis using exosomes, with higher accuracy and sensitivity than such conventional analysis. Also, the exosome-like vesicle according to the present invention contain a high concentration of miRNAs and is useful for the development of nucleic acid medicine.

### BRIEF DESCRIPTION OF DRAWINGS

FIG. 1 shows domain structures of various Argonaute protein mutants.

FIG. 2 shows results of Western blot analysis confirming various Argonaute protein mutants in cells and in extracellular vesicles.

FIG. 3 shows results of immunoprecipitation of Flag-Ago2-FL-Vpr or Flag-MID-PIWI-Vpr with an anti-Flag antibody.

FIG. 4 is a graph showing relative quantification values of miRNA-let7a-5p co-precipitated with Flag-Ago2-FL-Vpr or Flag-MID-PIWI-Vpr.

FIG. 5 shows results of Western blot analysis confirming Flag-Ago2-FL-Vpr or Flag-MID-PIWI-Vpr contained in cells and in extracellular vesicles.

FIG. 6 is a graph showing relative quantification values of miRNA-let7a-5p in extracellular vesicle fractions obtained from HEK293T cells co-expressing Flag-Ago2-FL-Vpr or Flag-MID-PIWI-Vpr and EPN-01.

FIG. 7 is a graph showing relative quantification values of miR-92a-3p in extracellular vesicle fractions obtained from HEK293T cells co-expressing Flag-MID-PIWI-Vpr and EPN-01.

FIG. 8 is a graph showing relative quantification values of miR-191-5p in extracellular vesicle fractions obtained from HEK293T cells co-expressing Flag-MID-PIWI-Vpr and EPN-01.

FIG. 9 is a graph showing relative quantification values of miR-126-5p in extracellular vesicle fractions obtained from HEK293T cells co-expressing Flag-MID-PIWI-Vpr and EPN-01.

FIG. 10 is a graph showing relative quantification values of miR-10b-5p in extracellular vesicle fractions obtained from HEK293T cells co-expressing Flag-MID-PIWI-Vpr and EPN-01.

FIG. 11 is a diagram showing results of Western blot analysis confirming changes in the expression levels of HIF1α, EPN-01, and MID-PIWI-Vpr due to CoCl₂ treatment.

FIG. 12 is a graph showing changes due to CoCl₂ treatment in the expression level of miR-210 in HEK293T cells transfected with or without Flag-MID-PIWI-Vpr and EPN-01.

FIG. 13 is a graph showing changes due to CoCl₂ treatment in the expression level of miR-1303 in HEK293T cells transfected with or without Flag-MID-PIWI-Vpr and EPN-01.

FIG. 14 is a graph showing changes due to CoCl₂ treatment in the content of miR-210 in extracellular vesicle fractions obtained from HEK293T cells transfected or not transfected with Flag-MID-PIWI-Vpr and EPN-01.

FIG. 15 is a graph showing vesicle concentrations and particle size distributions in extracellular vesicle fractions obtained from HEK293T cells co-expressing Flag-MID-PIWI-Vpr and EPN-01, HEK293T cells co-expressing EGFP and EPN-01, or untransfected HEK293T cells.

FIG. 16 is a graph showing the particle sizes (average values) of extracellular vesicles obtained from HEK293T cells co-expressing Flag-MID-PIWI-Vpr and EPN-01, HEK293T cells co-expressing EGFP and EPN-01, or untransfected HEK293T cells.

### DESCRIPTION OF EMBODIMENTS

Hereinafter, the present invention will be described in detail, but the present invention is not limited to the embodiments described herein.

According to a first embodiment, the present invention is a method for noninvasively acquiring a microRNA from a cell, the method comprising: (1) introducing a nucleic acid coding a microRNA-binding protein and a nucleic acid coding a vesicle-forming protein into the cell, wherein the microRNA-binding protein comprises a first portion consisting of an MID domain and a PIWI domain of an Argonaute protein and a second portion consisting of a virus protein R, and the vesicle-forming protein comprises: a palmitoylation or myristoylation signal or a pleckstrin homology domain; a self-assembling domain; an ESCRT or ESCRT-related factor-binding domain; and a Gag p6 domain; thereby producing an exosome-like vesicle including a microRNA, (2) collecting an extracellular fluid of the cell, and (3) extracting the microRNA from the extracellular fluid.

In the method of the present embodiment, a nucleic acid coding a microRNA-binding protein and a nucleic acid coding a vesicle-forming protein are introduced into a cell.

A “microRNA” (also referred to as “miRNA”) is a small, single-stranded non-coding RNA having a length of about 21 to 25 bases, and more than 20,000 types have been identified thus far (http://mirbase.org/). The miRNA in the present embodiment is not particularly limited, and may be any miRNA expressed in any cell. Also, the miRNA in the present embodiment can be not only a known one but also an unknown one. The miRNA in the present embodiment means a final miRNA product, and does not include intermediate products such as a pri-miRNA or a pre-miRNA.

The type of the “cell” in the present embodiment is not particularly limited, and can be, for example, a dendritic cell, a T cell, a B cell, a neuron, a stem cell, a cancer cell, or a primary culture cell or an established cell line derived therefrom. That is, the cell in the present embodiment may be either an in vivo cell or an in vitro cell. Also, the organism from which the cell is derived is not particularly limited either, and can be any vertebrate, and is preferably a mammal such as a mouse, a rat, a rabbit, a pig, a cow, a goat, a monkey, or a human, and is particularly preferably a human.

The “microRNA-binding protein” in the present embodiment includes a first portion consisting of an MID domain and a PIWI domain of an Argonaute protein and a second portion consisting of a virus protein R.

The “Argonaute protein” (hereinafter also referred to as “Ago”) is a protein that binds to a miRNA to form an RNA-induced silencing complex (RISC), and is composed of four characteristic domains: an N domain, a PAZ domain, an MID domain, and a PIWI domain, and two linker domains (L1, L2). The MID domain and the PIWI domain of the Ago used in the present embodiment may be derived from any protein of the Ago family, and are preferably derived from Ago1, Ago2, Ago3, or Ago4, and particularly preferably from Ago2. Also, the MID domain and the PIWI domain of the Ago used in the present embodiment may be derived from any vertebrate, and are preferably from a mammal, and are particularly preferably from a human. The amino acid sequence of human Ago2 (SEQ ID NO: 7) is shown below.

MYSGAGPALAPPAPPPPIQGYAFKPPPRPDFGTSGRTIKLQANFFEMDIP
KIDIYHYELDIKPEKCPRRVNREIVEHMVQHEKTQIFGDRKPVFDGRKNL
YTAMPLPIGRDKVELEVTLPGEGKDRIFKVSIKWVSCVSLQALHDALSGR
LPSVPFETIQALDVVMRHLPSMRYTPVGRSFFTASEGCSNPLGGGREVWF
GFHQSVRPSLWKMMLNIDVSATAFYKAQPVIEFVCEVLDFKSIEEQQKPL
TDSQRVKFTKEIKGLKVEITHCGQMKRKYRVCNVTRRPASHQTFPLQQES
GQTVECTVAQYFKDRHKLVLRYPHLPCLQVGQEQKHTYLPLEVCNIVAGQ
RCIKKLTDNQTSTMIRATARSAPDRQEEISKLMRSASFNTDPYVREFGIM
VKDEMTDVTGRVLQPPSILYGGRNKAIATPVQGVWDMRNKQFHTGIEIKV
WAIACFAPQRQCTEVHLKSFTEQLRKISRDAGMPIQGQPCFCKYAQGADS
VEPMFRHLKNTYAGLQLVVVILPGKTPVYAEVKRVGDTVLGMATQCVQMK
NVQRTTPQTLSNLCLKINVKLGGVNNILLPQGRPPVFQQPVIFLGADVTH
PPAGDGKKPSIAAVVGSMDAHPNRYCATVRVQQHRQEIIQDLAAMVRELL
IQFYKSTRFKPTRIIFYRDGVSEGQFQQVLHHELLAIREACIKLEKDYQP
GITFIVVQKRHHTRLFCTDKNERVGKSGNIPAGTTVDTKITHPTEFDFYL
CSHAGIQGTSRPSHYHVLWDDNRFSSDELQILTYQLCHTYVRCTRSVSIP
APAYYAHLVAFRARYHLVDKEHDSAEGSHTSGQSNGRDHQALAKAVQVHQ
DTLRTMYF

The “virus protein R” (hereinafter also referred to as “Vpr”) is a type of accessory protein specific to primate immunodeficiency viruses such as human immunodeficiency viruses (HIV) or simian immunodeficiency viruses (SIV), and interacts with the p6 domain of the structural protein Gag of the viruses. The Vpr used in the present embodiment may be derived from any primate immunodeficiency virus, and is preferably derived from an HIV, and particularly preferably from HIV-1. The amino acid sequence of HIV-1 Vpr (SEQ ID NO: 8) is shown below.

MEQAPEDQGPQREPHNEWTLELLEELKREAVRHFPRPWLHGLGQHIYETY
GDTWAGVEAIIRILQQLLFIHFRIGCQHSRIGIIQQRRARRNGASRS

The information of amino acid sequences of Ago and Vpr and nucleic acid sequences coding therefor can be obtained from a predetermined database. For example, for human Ago2, NP_036286.2 (GenBank) and NM_012154.5 (GenBank) are available. For HIV-1 Vpr, NP_057852.2 (GenBank) and NC_001802 (GenBank) (5105 to 5396) are available.

The microRNA-binding protein in the present embodiment most preferably comprises an amino acid sequence consisting of the MID domain and the PIWI domain of human Ago2 and HIV-1-derived Vpr (SEQ ID NO: 1).

MicroRNA-binding protein
(SEQ ID NO: 1)
IEIKVWAIACFAPQRQCTEVHLKSFTEQLRKISRDAGMPIQGQPCFCKYA
QGADSVEPMFRHLKNTYAGLQLVVVILPGKTPVYAEVKRVGDTVLGMATQ
CVQMKNVQRTTPQTLSNLCLKINVKLGGVNNILLPQGRPPVFQQPVIFLG
ADVTHPPAGDGKKPSIAAVVGSMDAHPNRYCATVRVQQHRQEIIQDLAAM
VRELLIQFYKSTRFKPTRIIFYRDGVSEGQFQQVLHHELLAIREACIKLE
KDYQPGITFIVVQKRHHTRLFCTDKNERVGKSGNIPAGTTVDTKITHPTE
FDFYLCSHAGIQGTSRPSHYHVLWDDNRFSSDELQILTYQLCHTYVRCTR
SVSIPAPAYYAHLVAFRARYHLVDKEHDSAEGSHTSGQSNGRDHQALAKA
VQVHQDTLRTMYFASRLEDIGGGGGGEQAPEDQGPQREPHNEWTLELLEE
LKREAVRHFPRPWLHGLGQHIYETYGDTWAGVEAIIRILQQLLFIHFRIG
CQHSRIGIIQQRRARRNGASRS

The microRNA-binding protein in the present embodiment may include a protein consisting of an amino acid sequence having an identity of 80% or more, preferably 90% or more, more preferably about 95% or more with the above amino acid sequences registered in the database, as long as binding activity to miRNA equivalent to the MID and PIWI domains of Ago and to the p6 domain of Gag equivalent to Vpr is maintained. The amino acid sequence identity can be determined by using sequence analysis software or by using a program commonly used in the art (FASTA, BLAST, etc.).

The “vesicle-forming protein” in the present embodiment includes a palmitoylation or myristoylation signal or a pleckstrin homology (PH) domain, a self-assembling domain, an ESCRT or ESCRT-related factor-binding domain, and a Gag p6 domain. A vesicle-forming protein that can be used in the present embodiment is disclosed, for example, as Enveloped Protein Nanocage (EPN) in WO 2016/138525, and specific examples thereof include, but are not limited to, EPN-01 (SEQ ID NO: 2), EPN-03 (SEQ ID NO: 3), EPN-07 (SEQ ID NO: 4), EPN-08 (SEQ ID NO: 5), and EPN-18 (SEQ ID NO: 6). The vesicle-forming protein in the present embodiment is preferably EPN-01 (SEQ ID NO: 2).

EPN-01
(SEQ ID NO: 2)
MGARASGSKSGSGSDSGSKIEELFKKHKIVAVLRANSVEEAKKKALAVFL
GGVHLIEITFTVPDADTVIKELSFLKEMGAIIGAGTVTSVEQCRKAVESG
AEFIVSPHLDEEISQFCKEKGVFYMPGVMTPTELVKAMKLGHTILKLFPG
EVVGPQFVKAMKGPFPNVKFVPTGGVNLDNVCEWFKAGVLAVGVGSALVK
GTPVEVAEKAKAFVEKIRGCTEQKLISEEDLQSRPEPTAPPEESFRSGVE
TTTPPQKQEPIDKELYPLTSLRSLFGNDPSSQ
EPN-03
(SEQ ID NO: 3)
MGAQFSGSKSGSGSDSGSKIEELFKKHKIVAVLRANSVEEAKKKALAVFL
GGVHLIEITFTVPDADTVIKELSFLKEMGAIIGAGTVTSVEQCRKAVESG
AEFIVSPHLDEEISQFCKEKGVFYMPGVMTPTELVKAMKLGHTILKLFPG
EVVGPQFVKAMKGPFPNVKFVPTGGVNLDNVCEWFKAGVLAVGVGSALVK
GTPVEVAEKAKAFVEKIRGCTEQKLISEEDLQSRPEPTAPPEESFRSGVE
TTTPPQKQEPIDKELYPLTSLRSLFGNDPSS
EPN-07
(SEQ ID NO: 4)
MGCIKSKRKDNLNGSKSGSGSDSGSKIEELFKKHKIVAVLRANSVEEAKK
KALAVFLGGVHLIEITFTVPDADTVIKELSFLKEMGAIIGAGTVTSVEQC
RKAVESGAEFIVSPHLDEEISQFCKEKGVFYMPGVMTPTELVKAMKLGHT
ILKLFPGEVVGPQFVKAMKGPFPNVKFVPTGGVNLDNVCEWFKAGVLAVG
VGSALVKGTPVEVAEKAKAFVEKIRGCTEQKLISEEDLQSRPEPTAPPEE
SFRSGVETTTPPQKQEPIDKELYPLTSLRSLEGNDPSS
EPN-08
(SEQ ID NO: 5)
MGCTLSAEERAALGSKSGSGSDSGSKIEELFKKHKIVAVLRANSVEEAKK
KALAVFLGGVHLIEITFTVPDADTVIKELSFLKEMGAIIGAGTVTSVEQC
RKAVESGAEFIVSPHLDEEISQFCKEKGVFYMPGVMTPTELVKAMKLGHT
ILKLFPGEVVGPQFVKAMKGPFPNVKFVPTGGVNLDNVCEWFKAGVLAVG
VGSALVKGTPVEVAEKAKAFVEKIRGCTEQKLISEEDLQSRPEPTAPPEE
SFRSGVETTTPPQKQEPIDKELYPLTSLRSLF
EPN-18
(SEQ ID NO: 6)
MHGLQDDPDLQALLKGSQLLKVKSSSWRRERFYKLQEDCKTIWQESRKVM
RSPESQLFSIEDIQEVRMGHRTEGLEKFARDIPEDRCFSIVFKDQRNTLD
LIAPSPADAQHWVQGLRKIIHHSGSMDQRQKGSGSKIEELFKKHKIVAVL
RANSVEEAKKKALAVFLGGVHLIEITFTVPDADTVIKELSFLKEMGAIIG
AGTVTSVEQCRKAVESGAEFIVSPHLDEEISQFCKEKGVFYMPGVMTPTE
LVKAMKLGHTILKLFPGEVVGPQFVKAMKGPFPNVKFVPTGGVNLDNVCE
WFKAGVLAVGVGSALVKGTPVEVAEKAKAFVEKIRGCTEQKLISEEDLQS
RPEPTAPPEESFRSGVETTTPPQKQEPIDKELYPLTSLRSLFGNDPSSQ

The vesicle-forming protein in the present embodiment can include a protein consisting of an amino acid sequence having an identity of 80% or more, preferably 90% or more, more preferably about 95% or more with the above amino acid sequences of the EPN subunits disclosed in the above publication, as long as activities of the EPN subunits are maintained (that is, nanocages can be formed by self-assembly to constitute extracellular vesicles).

The microRNA-binding protein and the vesicle-forming protein in the present embodiment may comprise an epitope tag, such as Myc, HA, or FLAG, added to the N-terminus and/or the C-terminus thereof.

A nucleic acid coding the microRNA-binding protein and a nucleic acid coding the vesicle-forming protein can be prepared by any conventionally known genetic engineering method using the sequences designed according to the above. Also, these nucleic acids may be introduced into a cell by well-known methods in the art, e.g., the nucleic acids may be cloned into an expression vector and thereby introduced into a cell. The type of the expression vector is not particularly limited, and the expression vector may be either a virus vector or a non-virus vector, and may be, e.g., a virus vector such as a herpesvirus vector, an adenovirus vector, a lentivirus vector, or a retrovirus vector, or a plasmid vector such as pCMV or pCAG.

When the microRNA-binding protein and the vesicle-forming protein are expressed in cells, microRNA-binding protein-miRNA complexes are stored in nanocages composed of the vesicle-forming proteins, and an exosome-like vesicles are formed and released outside the cell. The “exosome-like vesicle” in the present embodiment refers to nanoscale extracellular vesicles that are similar in structure and composition to exosomes.

Next, an extracellular fluid of the cell is collected. If the cell is an in vitro cell, the extracellular fluid may be a culture supernatant, and if the cell is an in vivo cell, the extracellular fluid may be a biological fluid. Examples of the biological fluid include, but are not limited to, blood, plasma, serum, saliva, and urine.

Next, miRNA is extracted from the extracellular fluid. The miRNA can be extracted by any established procedures, e.g., extracellular vesicles can be collected by ultracentrifugation and miRNAs can be isolated by purification methods such as the Boom method. Numerous miRNA extraction kits based on the Boom method are commercially available, and such commercially available products can also be used in the method of the present embodiment. Examples of a preferable commercially available products include High Pure miRNA Isolation Kit (Roche Diagnostics), miRNeasy Mini Kit (Qiagen), and mirVana™ miRNA Isolation Kit (Thermo Fisher Scientific).

The method of the present embodiment can acquire extracellular fluid containing high concentrations of miRNAs, enabling noninvasive, highly accurate, and highly sensitive diagnosis.

According to a second embodiment, the present invention is an exosome-like vesicle comprising (a) a microRNA, (b) a microRNA-binding protein comprising a first portion consisting of an MID domain and a PIWI domain of an Argonaute protein and a second portion consisting of a virus protein R, and (c) a nanocage composed of a vesicle-forming protein comprising an amino acid sequence having at least 90% identity to an amino acid sequence selected from the group consisting of SEQ ID NOs: 2 to 6.

The “exosome-like vesicle,” “microRNA,” “Argonaute protein,” “viral protein R,” “microRNA-binding protein,” and “vesicle-forming protein” in the present embodiment are as defined in the first embodiment.

The exosome-like vesicle of the present embodiment can further comprise (d) a membrane fusion protein. The “membrane fusion protein” means a protein that causes fusion between homogeneous or heterologous cells or membrane vesicles. The membrane fusion protein in the present embodiment is not particularly limited, and it is preferably an enveloped virus-derived membrane fusion protein, and examples thereof include vesicular stomatitis virus G protein (VSV-G), herpes simplex virus glycoprotein B (gB), and a recombinant thereof.

The exosome-like vesicle of the present embodiment can be acquired from extracellular fluid of cells into which a nucleic acid coding a microRNA-binding protein and a nucleic acid coding a vesicle-forming protein are introduced by the procedure of the method of the first embodiment.

The exosome-like vesicle of the present embodiment can contain a miRNA at a higher concentration than a natural exosome. Because of this, the exosome-like vesicle of the present embodiment is useful for diagnosis of a disease and development of a nucleic acid drug.

### EXAMPLES

The present invention will be further described with reference to Examples shown below. These by no means limit the present invention.

1. Search for Argonaute Protein Mutants that can be Stored in Extracellular Vesicles

In the present Example, EPN-01 (SEQ ID NO: 2) was used as the vesicle-forming protein, and human Ago2 was used as the Argonaute protein. Since the nanocage formed by EPN-01 was 20 nm in diameter, it was assumed that it would be difficult for the full-length Ago2 to be encapsulated therein. Thus, mutants having different sizes were prepared from Ago2, and they were tested to see if these could be encapsulated in the nanocage.

Expression vectors for Ago2 mutants of the full-length Ago2 (amino acids 1 to 860), the PAZ domain to the PIWI domain of Ago2 (amino acids 227 to 860), the L2 domain to the PIWI domain of Ago2 (amino acids 347 to 860), and the MID domain to the PIWI domain of Ago2 (amino acids 446 to 860), with a FLAG tag added to the N-terminus and HIV-1-derived Vpr (SEQ ID NO: 1) added to the C-terminus (Flag-Ago2-FL-Vpr, Flag-PAZ-PIWI-Vpr, Flag-L2-PIWI-Vpr, and Flag-MID-PIWI-Vpr, respectively, FIG. 1), were prepared by the following procedures.

We commissioned VectorBuilder, Inc., to synthesize a plasmid VB200705-1156knk (SEQ ID NO: 9), comprising sequences coding EPN-01 and Myc-EGFP-Vpr. Inverse PCR was performed using VB200705-1156knk as a template with the following primers 1 and 2. The resulting amplified product was cleaved at the EcoRI site and ligated to obtain pRP-Myc-EGFP-Vpr. PCR was performed using pcDNA3.2-5′F-Ago2, a plasmid comprising a sequence coding full-length human Ago2 with a Flag tag added to the N-terminus, as a template with the following primers 3 and 4. The resulting amplification product was inserted into the XbaI site of pRP-Myc-EGFP-Vpr by In-Fusion cloning to obtain pRP-Flag-Ago2-FL-Vpr, a plasmid comprising a sequence coding Flag-Ago2-FL-Vpr. PCR was performed using pRP-Flag-Ago2-FL-Vpr as a template with the following primers 5 and 6. The resulting amplified product was inserted into the Nhel/HindIII site of pEGFP-C2 (Clontech) by In-Fusion cloning to obtain pCMV-Flag-Ago2-FL-Vpr.

PCR was performed using pcDNA3.2-5′F-Ago2 as a template with the following primers 7 and 10, 8 and 10, or 9 and 10. The resulting amplified product was inserted into the Nhel/XbaI site of pCMV-Flag-Ago2-FL-Vpr by In-Fusion cloning to obtain pCMV-Flag-PAZ-PIWI-Vpr, pCMV-Flag-L2-PIWI-Vpr, and pCMV-Flag-MID-PIWI-Vpr.

TABLE 1
Primer sets for constructing Ago2 mutant
expression vectors
[Table 1]

| | | | SEQ |
|---|---|---|---|
| | Primer | | ID |
| No. | name | Sequence (5′→3′) | NO |
| 1 | EcoR1-Myc- | GCGAATTCTTAATTAATCTAGACA | 10 |
| | EGFP_F | TGGA | |
| 2 | EF1A- | GCGAATTCAGCCTGCTTTTTTGTA | 11 |
| | EcoR1_R | CAAA | |
| 3 | VLP-Flag- | TTAATTAATCTAGACATGGATTAC | 12 |
| | Ago2_F | AAAGACGATGACGATAAGATGTAC | |
| | | TCGGGAGCCGGCCC | |
| 4 | VLP-Ago2_R | ATCCTCGAGTCTAGAAGCAAAGTA | 13 |
| | | CATGGTGCGCA | |
| 5 | pΔEGFP_ | GTCAGATCCGCTAGCGCCACCATG | 14 |
| | Flag_F | GATTACAAAGACGATGA | |
| 6 | PRP_ | CAGAATTCGAAGCTTTTTGTACAA | 15 |
| | pΔEGFP- | GAAAGCTGGGT | |
| | C2_R | | |
| 7 | pdEGFP_ | GTCAGATCCGCTAGCGCCACCATG | 16 |
| | Flag-Ago2 | GATTACAAAGACGATGACGATGCA | |
| | _Paz- | CAGCCAGTAATCGAGTT | |
| | PIWI_F | | |
| 8 | pdEGFP_ | GTCAGATCCGCTAGCGCCACCATG | 17 |
| | Flag-Ago2 | GATTACAAAGACGATGACGATGCA | |
| | _L2-PIWI_ | GGACAAAGATGTATTAA | |
| | F | | |
| 9 | pdEGFP_ | GTCAGATCCGCTAGCGCCACCATG | 18 |
| | Flag-Ago2- | GATTACAAAGACGATGACGATAAG | |
| | dN-PAZ_F | ATCGAGATCAAGGTGTGGGC | |
| 10 | Ago2-Xba- | TCCGATATCCTCGAGAGCAAAGTA | 19 |
| | Vpr_R | CATGGTGCGCA | |

The amino acid sequences of Flag-Argonaute protein mutant-Vpr expressed from the plasmid vectors prepared above are shown below.

Flag-Ago2-FL-Vpr
(SEQ ID NO: 23)
MDYKDDDDKMYSGAGPALAPPAPPPPIQGYAFKPPPRPDFGTSGRTIKLQ
ANFFEMDIPKIDIYHYELDIKPEKCPRRVNREIVEHMVQHFKTQIFGDRK
PVFDGRKNLYTAMPLPIGRDKVELEVTLPGEGKDRIFKVSIKWVSCVSLQ
ALHDALSGRLPSVPFETIQALDVVMRHLPSMRYTPVGRSFFTASEGCSNP
LGGGREVWFGFHQSVRPSLWKMMLNIDVSATAFYKAQPVIEFVCEVLDFK
SIEEQQKPLTDSQRVKFTKEIKGLKVEITHCGQMKRKYRVCNVTRRPASH
QTFPLQQESGQTVECTVAQYFKDRHKLVLRYPHLPCLQVGQEQKHTYLPL
EVCNIVAGQRCIKKLTDNQTSTMIRATARSAPDRQEEISKLMRSASFNTD
PYVREFGIMVKDEMTDVTGRVLQPPSILYGGRNKAIATPVQGVWDMRNKQ
FHTGIEIKVWAIACFAPQRQCTEVHLKSFTEQLRKISRDAGMPIQGQPCF
CKYAQGADSVEPMFRHLKNTYAGLQLVVVILPGKTPVYAEVKRVGDTVLG
MATQCVQMKNVQRTTPQTLSNLCLKINVKLGGVNNILLPQGRPPVFQQPV
IFLGADVTHPPAGDGKKPSIAAVVGSMDAHPNRYCATVRVQQHRQEIIQD
LAAMVRELLIQFYKSTRFKPTRIIFYRDGVSEGQFQQVLHHELLAIREAC
IKLEKDYQPGITFIVVQKRHHTRLFCTDKNERVGKSGNIPAGTTVDTKIT
HPTEFDFYLCSHAGIQGTSRPSHYHVLWDDNRFSSDELQILTYQLCHTYV
RCTRSVSIPAPAYYAHLVAFRARYHLVDKEHDSAEGSHTSGQSNGRDHQA
LAKAVQVHQDTLRTMYFASRLEDIGGGGGGEQAPEDQGPQREPHNEWTLE
LLEELKREAVRHFPRPWLHGLGQHIYETYGDTWAGVEAIIRILQQLLFIH
FRIGCQHSRIGIIQQRRARRNGASRS
Flag-PAZ-PIWI-Vpr
(SEQ ID NO: 24)
MDYKDDDDKAQPVIEFVCEVLDFKSIEEQQKPLTDSQRVKFTKEIKGLKV
EITHCGQMKRKYRVCNVTRRPASHQTFPLQQESGQTVECTVAQYFKDRHK
LVLRYPHLPCLQVGQEQKHTYLPLEVCNIVAGQRCIKKLTDNQTSTMIRA
TARSAPDRQEEISKLMRSASENTDPYVREFGIMVKDEMTDVTGRVLQPPS
ILYGGRNKAIATPVQGVWDMRNKQFHTGIEIKVWAIACFAPQRQCTEVHL
KSFTEQLRKISRDAGMPIQGQPCFCKYAQGADSVEPMFRHLKNTYAGLQL
VVVILPGKTPVYAEVKRVGDTVLGMATQCVQMKNVQRTTPQTLSNLCLKI
NVKLGGVNNILLPQGRPPVFQQPVIFLGADVTHPPAGDGKKPSIAAVVGS
MDAHPNRYCATVRVQQHRQEIIQDLAAMVRELLIQFYKSTREKPTRIIFY
RDGVSEGQFQQVLHHELLAIREACIKLEKDYQPGITFIVVQKRHHTRLFC
TDKNERVGKSGNIPAGTTVDTKITHPTEFDFYLCSHAGIQGTSRPSHYHV
LWDDNRESSDELQILTYQLCHTYVRCTRSVSIPAPAYYAHLVAFRARYHL
VDKEHDSAEGSHTSGQSNGRDHQALAKAVQVHQDTLRTMYFASRLEDIGG
GGGGEQAPEDQGPQREPHNEWTLELLEELKREAVRHFPRPWLHGLGQHIY
ETYGDTWAGVEAIIRILQQLLFIHFRIGCQHSRIGIIQQRRARRNGASRS
Flag-L2-PIWI-Vpr
(SEQ ID NO: 25)
MDYKDDDDKAGQRCIKKLTDNQTSTMIRATARSAPDRQEEISKLMRSASF
NTDPYVREFGIMVKDEMTDVTGRVLQPPSILYGGRNKAIATPVQGVWDMR
NKQFHTGIEIKVWAIACFAPQRQCTEVHLKSFTEQLRKISRDAGMPIQGQ
PCFCKYAQGADSVEPMFRHLKNTYAGLQLVVVILPGKTPVYAEVKRVGDT
VLGMATQCVQMKNVQRTTPQTLSNLCLKINVKLGGVNNILLPQGRPPVFQ
QPVIFLGADVTHPPAGDGKKPSIAAVVGSMDAHPNRYCATVRVQQHRQEI
IQDLAAMVRELLIQFYKSTRFKPTRIIFYRDGVSEGQFQQVLHHELLAIR
EACIKLEKDYQPGITFIVVQKRHHTRLFCTDKNERVGKSGNIPAGTTVDT
KITHPTEFDFYLCSHAGIQGTSRPSHYHVLWDDNRFSSDELQILTYQLCH
TYVRCTRSVSIPAPAYYAHLVAFRARYHLVDKEHDSAEGSHTSGQSNGRD
HQALAKAVQVHQDTLRTMYFASRLEDIGGGGGGEQAPEDQGPQREPHNEW
TLELLEELKREAVRHFPRPWLHGLGQHIYETYGDTWAGVEAIIRILQQLL
FIHFRIGCQHSRIGIIQQRRARRNGASRS
Flag-MID-PIWI-Vpr
(SEQ ID NO: 26)
MDYKDDDDKIEIKVWAIACFAPQRQCTEVHLKSFTEQLRKISRDAGMPIQ
GQPCFCKYAQGADSVEPMFRHLKNTYAGLQLVVVILPGKTPVYAEVKRVG
DTVLGMATQCVQMKNVQRTTPQTLSNLCLKINVKLGGVNNILLPQGRPPV
FQQPVIFLGADVTHPPAGDGKKPSIAAVVGSMDAHPNRYCATVRVQQHRQ
EIIQDLAAMVRELLIQFYKSTREKPTRIIFYRDGVSEGQFQQVLHHELLA
IREACIKLEKDYQPGITFIVVQKRHHTRLFCTDKNERVGKSGNIPAGTTV
DTKITHPTEFDFYLCSHAGIQGTSRPSHYHVLWDDNRESSDELQILTYQL
CHTYVRCTRSVSIPAPAYYAHLVAFRARYHLVDKEHDSAEGSHTSGQSNG
RDHQALAKAVQVHQDTLRTMYFASRLEDIGGGGGGEQAPEDQGPQREPHN
EWTLELLEELKREAVRHFPRPWLHGLGQHIYETYGDTWAGVEAIIRILQQ
LLFIHFRIGCQHSRIGIIQQRRARRNGASRS

PCR was performed using VB200705-1156knk (SEQ ID NO: 9) as a template with the following primers 11 and 12, so as to construct an EPN-01 expression vector. The resulting amplified product was cleaved at the KpnI site and ligated to obtain pRP-EPN-01. PCR was performed using pRP-EPN-01 as a template with the primer 6 above and primer 13 below. The resulting amplified product was inserted into the Nhel/HindIII site of pEGFP-C2 by In-Fusion cloning to obtain pCMV-EPN-01.

TABLE 2
Primer sets for constructing Ago2 mutant
expression vectors
[Table 2]

| | | | SEQ |
|---|---|---|---|
| | Primer | | ID |
| No. | name | Sequence (5′→3′) | NO |
| 11 | kpn1- | GCGGTACCACCCAGCTTTCTTGTA | 20 |
| | SV40_F | CAAA | |
| 12 | EPN01- | GCGGTACCTTACTGAGAAGACGGG | 21 |
| | kpn1_R | TCGTTAC | |
| 13 | pΔEGFP_ | GTCAGATCCGCTAGCGCCACCATG | 22 |
| | EPN01_F | GGTGCGCGTGC | |

HEK293T cells (ATCC) were placed in a 10 cm dish. The next day, each Ago2 mutant expression vector (5 μg) and the EPN-01 expression vector (10 μg) were transfected with Lipofectamine 2000 (Thermo Fisher Scientific), and after 6 hours, the medium was replaced with 10 ml of fresh medium. At 24 hours after transfection, the medium was collected and centrifuged at 200×g for 5 minutes at 4° C., at 1,000×g for 5 minutes at 4° C., and at 10,000×g for 30 minutes at 4° C. to collect the supernatant. The supernatant was layered on 2 ml of a 20% sucrose solution placed in an ultracentrifugation tube, and ultracentrifuged at 100,000×g for 90 minutes at 4° C. After that, the supernatant was discarded, and the pellet was gently washed with PBS. The liquid was removed, and then 100 μl of 1×SDS sample buffer (Tris-HCl (62.5 mM), pH 6.8, 20% glycerol, 2% SDS, 2.5% 2-mercaptoethanol) was added to the bottom without touching the wall of the tube to dissolve the pellet to obtain an extracellular-vesicle solution. The obtained solution was stored at −80° C. On the other hand, the HEK293T cells after collection of the medium were washed with PBS, collected, and 1/5 amount thereof was dissolved in 200 μl of 1× SDS sample buffer to obtain a cell solution.

The extracellular-vesicle solution and the cell solution were subjected to SDS-PAGE (10% acrylamide gel), and after electrophoresis, proteins were transferred to a PDVF membrane. After the transfer, the PDVF membrane was blocked with 3.5% skim milk for 30 minutes, washed with TBS-T (Tris-HCl (25 mM), NaCl (150 mM), 0.1% Tween 20) three times (30 minutes in total), and then incubated in a primary antibody solution overnight at 4° C. The primary antibody solution was removed, the PDVF membrane was washed with TBS-T three times (30 minutes in total), and then incubated in a secondary antibody solution for 1 hour at room temperature. The secondary antibody solution was removed, and the PDVF membrane was washed with TBS-T three times (30 minutes in total), and then a reaction for detection using ImmunoStar LD (Wako) was carried out, which was detected with LAS3000 (FUJIFILM). As the primary antibody, an anti-c-Myc antibody (anti-c-myc from mouse IgGIK [9E10] (11667203001, Roche) (1:1000 dilution)); an anti-Flag antibody (Monoclonal ANTI-FLAG™ M2 antibody produced in mouse (F3165, Sigma-Aldrich (1:1000 dilution)); and an anti-ß-actin antibody (B-Actin (13E5) Rabbit mAb (#4970, Cell Signaling Technology) (1:1000 dilution)) were used. As the secondary antibody, ECL™ anti-rabbit IgG (NA9340V, GE) (1:2000 dilution) and ECL™ anti-mouse IgG (NA9310V, GE) (1:2000 dilution) were used.

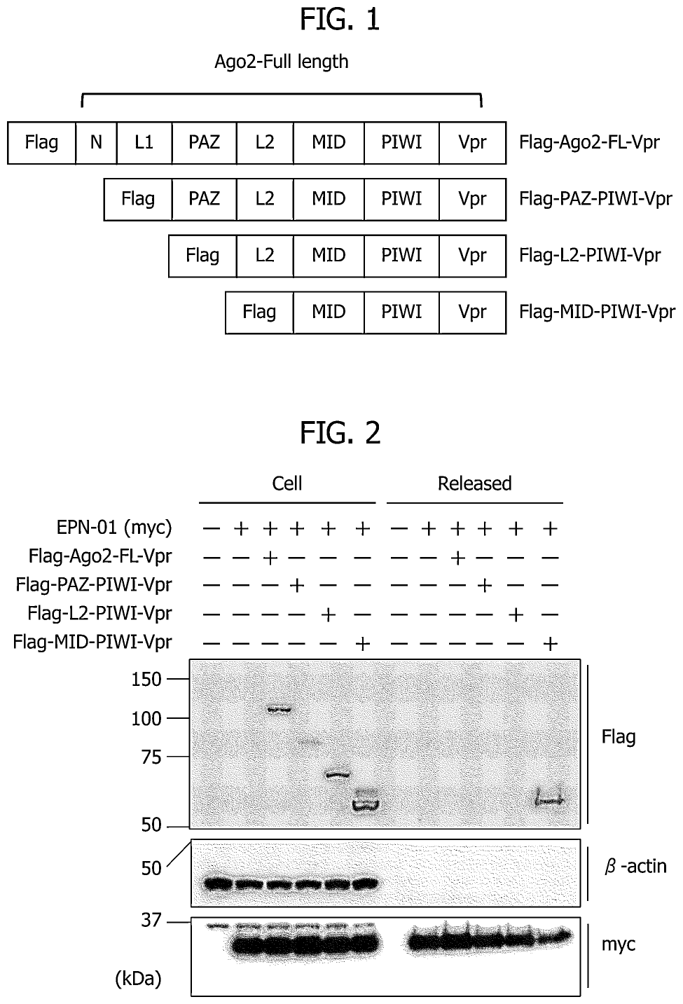

Results are shown in FIG. 2. Hereinafter, in the Figure, “Cell” indicates the results for the cell solution, and “Released” indicates the results for the extracellular-vesicle solution. Expression of the full-length Ago2 and all the Ago2 mutants was observed in the cell solution, but only the MID-PIWI-Vpr mutant was detected in the extracellular-vesicle solution. These results confirm that only the MID-PIWI-Vpr mutant can be incorporated into an EPN-01 nanocage.

2. miRNA Binding Activity of MID-PIWI-Vpr Mutant

Next, in order to examine the miRNA-binding activity of the MID-PIWI-Vpr mutant, the full-length Ago2 (Flag-Ago2-FL-Vpr) or Flag-MID-PIWI-Vpr was expressed in HEK293T cells by the same procedure as in 1 above. As a control, EGFP was expressed instead of the Ago2 mutant. At 24 hours after transfection, the cells were washed once with PBS, and 1 mL of Lysis Buffer (HEPES (20 mM), pH 7.5, NaCl (150 mM), NaF (50 mM), Na₃ VO₄ (1 mM), 1% Digitonin, phenylmethylsulfonyl fluoride (1 mM), Leupeptin (5 μg/ml), Aprotinin (5 μg/ml), Pepstatin A (3 μg/ml)) was added. The cell lysate was collected with a scraper and centrifuged at 15,000 rpm for 10 minutes at 4° C., and the supernatant was collected. A 50 μl aliquot was taken from the supernatant, and 50 μl of 2× SDS sample buffer was added thereto to obtain a cell solution sample before immunoprecipitation.

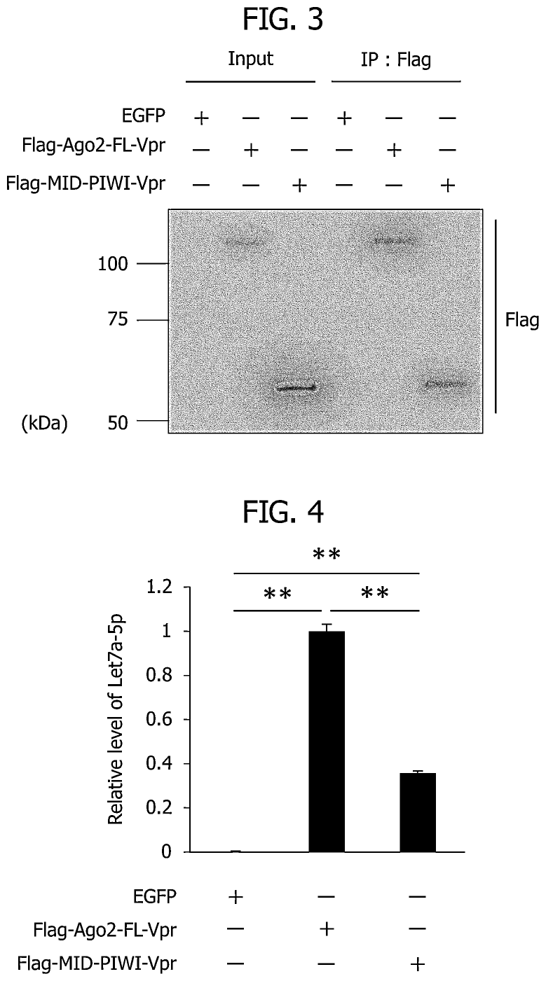

Dynabeads Protein G (Veritas), which was washed with Wash Buffer (HEPES (10 mM) pH 7.5, NaCl (150 mM), 0.1% Triton-X) and reacted with an anti-Flag-M2 antibody (Sigma-Aldrich, F1804, 1:800 dilution) for 30 minutes at room temperature in advance, was added to the supernatant, and was incubated and rotated for 1 hour at 4° C. The beads were washed three times with Wash Buffer, and then 120 μl of Elution Buffer was added, mixed, and incubated for 5 minutes at 4° C. After that, a 100 μl of the supernatant was collected, and 100 μl of 2× SDS sample buffer was added thereto to obtain an immunoprecipitate sample. The cell solution before immunoprecipitation (“Input” in FIG. 3) and the immunoprecipitate (“IP: Flag” in FIG. 3) were subjected to Western blotting in the same procedure as in 1 above to confirm that the Flag-Ago2-FL-Vpr and Flag-MID-PIWI-Vpr mutants were immunoprecipitated (FIG. 3).

Next, the miRNA was purified from the immunoprecipitate by using the mirVana™ miRNA Isolation Kit (final volume of 50 μl). Reverse transcription reactions were performed to prepare cDNA by using 5 μl of miRNA solution, TaqMan™ MicroRNA Reverse Transcription Kit (Thermo Fisher), and TaqMan™ MicroRNA Assays (Thermo Fisher). Furthermore, qPCR was performed with TaqMan™ Universal Master Mix II, no UNG (Thermo Fisher) TaqMan™ MicroRNA Assays (Thermo Fisher) to quantify a cDNA of miRNA-let7a-5p. The level of the miRNA was defined as a relative value when the amount of miRNA in the immunoprecipitate sample prepared from EGFP-expressing cells was set to 1.

Results are shown in FIG. 4. In the Figure, an asterisk (*) indicates p-values by one-way ANOVA with Tukey post-hoc test (** p<0.01). Error bars indicate standard deviations. The amount of miRNA-let7a-5p co-precipitated with Flag-MID-PIWI-Vpr was reduced as compared with the amount of miRNA-let7a-5p co-precipitated with Flag-Ago2-FL-Vpr, but was significantly greater than that of the negative control EGFP. These results indicate that the MID-PIWI-Vpr mutant binds to miRNA-let7a-5p.

3. Amount of miRNA-let7a-5p in Extracellular Vesicles Containing EPN-01/MID-PIWI-Vpr Mutant

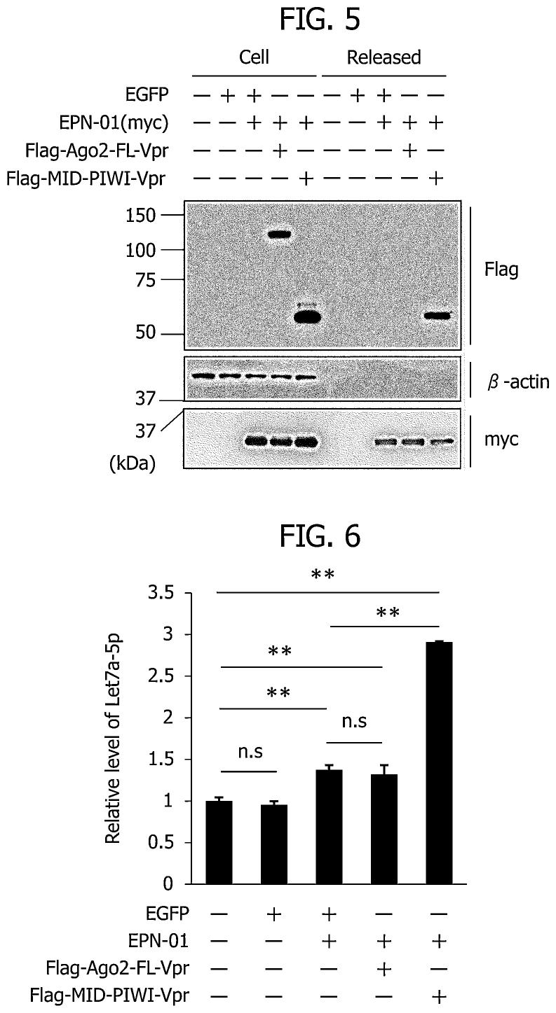

HEK293T cells co-expressing full-length Ago2 (Flag-Ago2-FL-Vpr) and EPN-01 or co-expressing Flag-MID-PIWI-Vpr and EPN-01 were prepared by the same procedure as in 1 above, and the cell solution and the extracellular-vesicle solution therefrom were subjected to Western blotting to confirm expression of proteins (FIG. 5). Only the MID-PIWI-Vpr mutant was detected from the extracellular-vesicle solution, as is the case with the result in FIG. 2.

Next, miRNA-let7a-5p in the extracellular-vesicle solution was purified and quantified by the same procedure as in 2 above. The level of the miRNA was defined as a relative value when the amount of the miRNA in the extracellular-vesicle solution prepared from untransfected cells was set to 1.

Results are shown in FIG. 6. In the Figure, an asterisk (*) indicates p-values by one-way ANOVA with Tukey post-hoc test (** p<0.01). Error bars indicate standard deviations. The yield of miRNA-let7a-5p from the extracellular-vesicle solution obtained from HEK293T cells co-expressing EPN-01 and Flag-MID-PIWI-Vpr was greatly increased. On the other hand, even when EPN-01 and Flag-Ago2-FL-Vpr were co-expressed, there was no significant increase in the yield of miRNA-let7a-5p as compared with the negative control in which EPN-01 and EGFP were co-expressed. These results indicate that co-expression of EPN-01 and MID-PIWI-Vpr mutant can increase miRNA-let7a-5p in an extracellular vesicle fraction.

4. Type and Amount of miRNA in EPN-01/MID-PIWI-Vpr Mutant-Containing Extracellular Vesicles

Next-generation sequencing small RNA-seq was used to comprehensively identify miRNAs present in the extracellular vesicle fraction obtained from HEK293T cells co-expressing EPN-01 and Flag-MID-PIWI-Vpr. The extracellular vesicle fraction obtained from HEK293T cells without transfection and only with medium replacement was prepared as a control. As a result, 186 miRNAs were identified from the control extracellular vesicle fraction, and 323 miRNAs were identified from the extracellular vesicle fraction obtained from the EPN-01/MID-PIWI-Vpr co-expressing cells. Also, 145 miRNAs were commonly present in both the extracellular vesicle fractions, 41 miRNAs were present only in the control extracellular vesicle fraction, and 178 miRNAs were present only in the extracellular vesicle fraction obtained from the EPN-01/MID-PIWI-Vpr co-expressing cells. These results confirm that the types of miRNAs detectable from an extracellular vesicle fraction were increased by co-expression of EPN-01/MID-PIWI-Vpr.

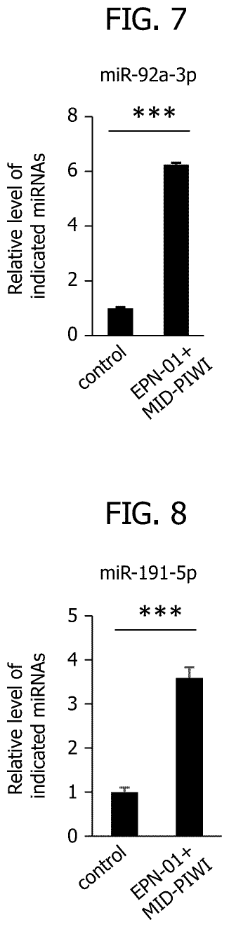

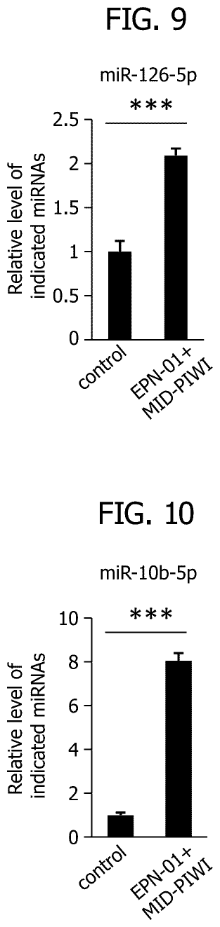

Next, miR-92a-3p, miR-191-5p, and miR-126-5p, which were commonly detected in large amounts in the control and the extracellular vesicle fraction obtained from the EPN-01/MID-PIWI-Vpr co-expressing cells, were quantified by RT-qPCR by the same procedure as in 2 above. Results are shown in FIGS. 7 to 9. In the Figures, an asterisk (*) indicates p-values by Student's t-test (*** p<0.001). Error bars indicate standard deviations. It was confirmed that the yields of all miRNAs were increased by co-expression of EPN-01/MID-PIWI-Vpr.

Furthermore, FIG. 10 shows the results regarding miR-10b-5p, which was found only in the extracellular vesicle fraction from EPN-01/MID-PIWI-Vpr co-expressing cells in small RNA-seq analysis. In the Figure, an asterisk (*) indicates p-values by Student's t-test (*** p <0.001). Error bars indicate standard deviations. RT-qPCR confirms that although miR-10b-5p was present in the control extracellular vesicle fraction, more than 8-fold more miR-10b-5p was present in the extracellular vesicle fraction from the EPN-01/MID-PIWI-Vpr co-expressing cells.

The above results indicate that co-expression of EPN-01 and MID-PIWI-Vpr mutant can increase the amount of a wide variety of miRNAs in the extracellular vesicle fraction, and it may be possible to analyze even trace amounts of miRNA that cannot be detected from normal exosomes.

### 5. Nondestructive Analysis of Cells Using EPN-01/MID-PIWI-Vpr Mutant-Containing Extracellular Vesicles

It is known that the expression of miR-210 is strongly induced under hypoxic conditions. One of the well-studied hypoxia signaling pathways is controlled by hypoxia-inducible factor (HIF). Under normoxic conditions, HIF1a is hydroxylated, bound to the E3 ligase, and degraded by the proteasome. In contrast, under hypoxic conditions, HIF1a is stabilized, translocates to the nucleus without degradation, forms a dimer with HIF1B, and promotes transcription of target genes including miR-210 (Genes Dev. 2004 Sep. 15;18(18):2183-94. doi: 10.1101/gad.1243304., Mol Cell. 2009 Sep. 24;35(6):856-67. doi: 10.1016/j.molcel.2009.09.006.).

Western blotting was performed on the cell solution and the extracellular-vesicle solution by the same procedure as in 1 above to confirm protein expression, except that CoCl₂ (50 μM) was added six hours after transfection of the MID-PIWI-Vpr expression vector and the EPN-01 expression vector to induce hypoxia. Anti-HIF-1α antibody [EP1215Y] (Abcam, ab51608, 1:1000 dilution) was used to detect HIF1α.

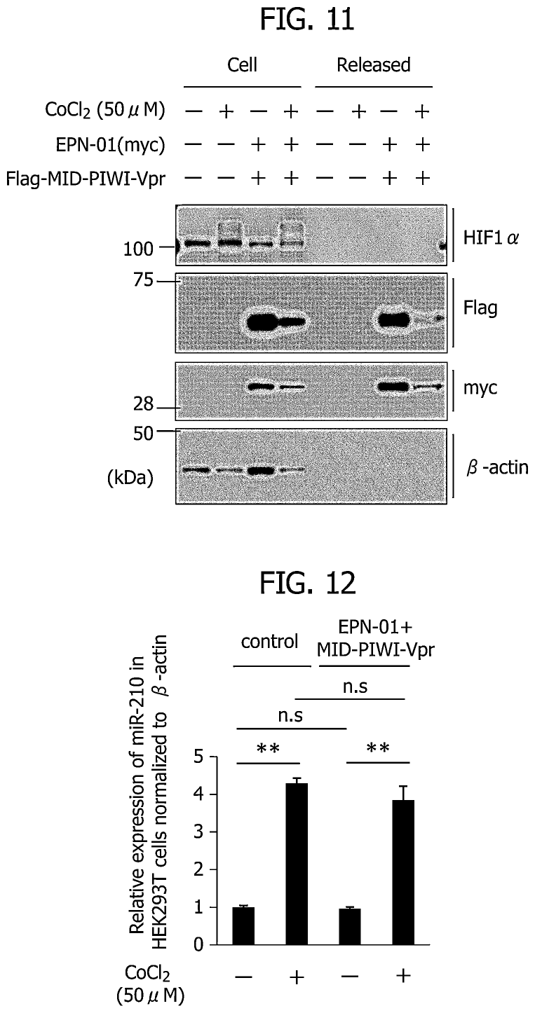

Results are shown in FIG. 11. Accumulation of HIF 1α was observed in cells treated with CoCl₂. These results are consistent with reports that HIF1a accumulates when hypoxia is induced by CoCl₂ treatment (Biol Res. 2019 Mar. 15;52(1): 12. doi: 10.1186/s40659-019-0221-z.). Also, the expression levels of EPN-01 and MID-PIWI-Vpr decreased in CoCl₂-treated cells, and these results are consistent with reports that protein production is inhibited under hypoxic conditions (Mol Cell Biol. 2006 May; 26(10):3955-65. doi: 10.1128/MCB.26.10.3955-3965.2006.). Similarly, the yields of EPN-01 and MID-PIWI-Vpr in the extracellular vesicle fraction also decreased.

Next, miR-210 in the cell solution and the extracellular-vesicle solution was purified and quantified by the same procedure as in 2 above. As controls, the cell solution and the extracellular-vesicle fraction obtained from HEK293T cells without transfection and only with medium replacement were prepared. In addition, for comparison, miR-1303, which has not been reported to be associated with a hypoxic condition, was similarly purified and quantified.

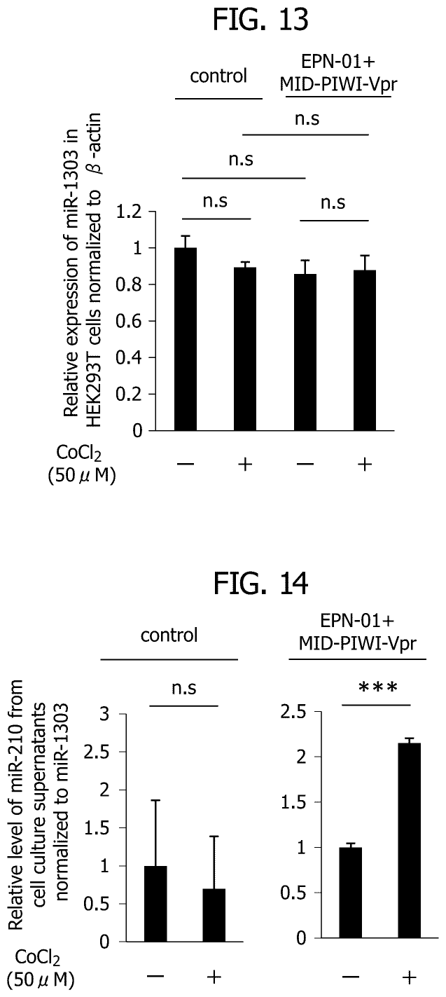

Quantification results of miR-210 in cells are shown in FIG. 12, and quantification results of miR-210 in cells are shown in FIG. 13. In the Figures, an asterisk (*) indicates p-values by one-way ANOVA with Tukey post-hoc test (** p<0.01). Error bars indicate standard deviations. CoCl₂ treatment increased the expression level of miR-210, but did not significantly change the expression level of miR-1303. Also, no effect of co-expression of EPN-01 and MID-PIWI-Vpr was observed on the expression of either miRNA.

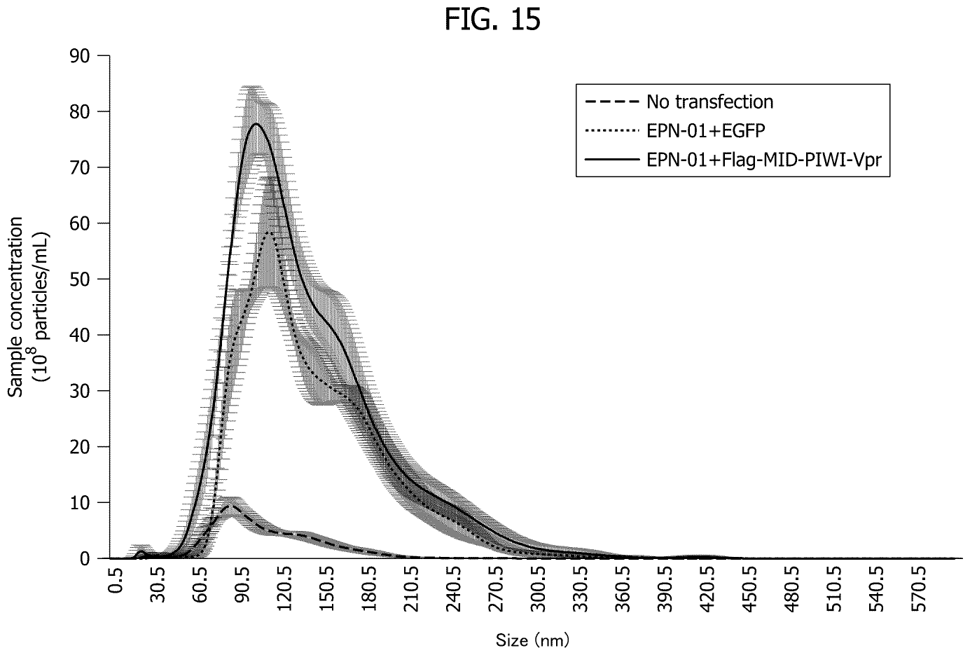

As confirmed above, CoCl₂ treatment reduced the expression levels of EPN-01 and MID-PIWI-Vpr in cells, which was assumed to reduce extracellular vesicles and miRNAs encapsulated therein. Thus, the quantification results of miR-210 normalized based on the quantification results of miR-1303 are shown in FIG. 14. In the Figure, an asterisk (*) indicates p-values by Student's t-test (*** p<0.001). Error bars indicate standard deviations. In the extracellular-vesicle fraction from control cells, no significant increase in miR-210 levels was observed due to CoCl₂ treatment. On the other hand, in the extracellular-vesicle fraction from cells co-expressing EPN-01 and MID-PIWI-Vpr, a significant increase in miR-210 levels was observed due to CoCl₂ treatment. These results indicate that co-expression of EPN-01 and MID-PIWI-Vpr enabled the detection of increased miR-210 expression due to CoCl₂ treatment with high accuracy.

The above results indicate that co-expression of EPN-01 and MID-PIWI-Vpr in cells enables miRNA analysis, without destroying the cells, with higher accuracy and sensitivity than conventional miRNA analysis using an exosome.

### 6. EPN-01/MID-PIWI-Vpr Co-Expression Increases Extracellular Vesicle Size and Production Volume

By the same procedure as in 1 above, HEK293T cells co-expressing EPN-01 and Flag-MID-PIWI-Vpr and HEK293T cells co-expressing EPN-01 and EGFP were prepared, and extracellular-vesicle fractions were obtained from the culture supernatant by sucrose cushion centrifugation. Untransfected HEK293T cells were used as a control. Nanoparticle tracking analysis (NTA) was performed under the following conditions by using Nanosite NS300 (Malvern Panalytical). The camera level was set to 16 for all recordings. The extracellular-vesicle fractions were diluted 1:100 to 1:1000 with PBS to prepare measurement samples in such a way as to have a number of particles of 1×10⁸ to 1× 109/ml. The camera focus was adjusted such that particles appeared as sharp individual dots. A 60-second image was recorded 5 times for each measurement sample. All data acquisition functions were set to automatic except the detection threshold was set to 8.

The particle size distributions and the concentrations of extracellular vesicles are shown in FIG. 15. Error bars indicate standard errors. Both the HEK293T cells co-expressing EPN-01 and Flag-MID-PIWI-Vpr and the HEK293T cells co-expressing EPN-01 and EGFP had increased numbers of extracellular vesicles as compared with the untransfected HEK293T cells. Also, both the HEK293T cells co-expressing EPN-01 and Flag-MID-PIWI-Vpr and the HEK293T cells co-expressing EPN-01 and EGFP had similar trends in the particle size distribution of extracellular vesicles.

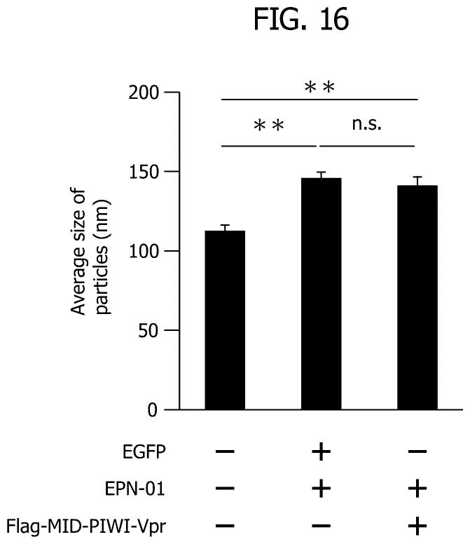

The average values of the particle sizes of extracellular vesicles are shown in FIG. 16. In the Figure, an asterisk (*) indicates p-values by one-way ANOVA with Tukey post-hoc test (** p<0.01). Error bars indicate standard errors. Both the HEK293T cells co-expressing EPN-01 and Flag-MID-PIWI-Vpr and the HEK293T cells co-expressing EPN-01 and EGFP tended to increase in the particle size of extracellular vesicles.

The above results indicate that extracellular vesicles that are larger than normal exosomes can be obtained at a high concentration from cells co-expressing EPN-01 and the MID-PIWI-Vpr mutant.

1. A method for noninvasively acquiring a microRNA from a cell, the method comprising:
(1) introducing a nucleic acid coding a microRNA-binding protein and a nucleic acid coding a vesicle-forming protein into the cell, wherein the microRNA-binding protein comprises a first portion consisting of an MID domain and a PIWI domain of an Argonaute protein and a second portion consisting of a virus protein R, and the vesicle-forming protein comprises: a palmitoylation or myristoylation signal or a pleckstrin homology domain; a self-assembling domain; an ESCRT or ESCRT-related factor-binding domain; and a Gag p6 domain; thereby producing an exosome-like vesicle comprising a microRNA,
(2) collecting an extracellular fluid of the cell, and
(3) extracting the micro-RNA from the extracellular fluid.

2. The method according to claim 1, wherein the microRNA-binding protein comprises an amino acid sequence having at least 90% identity to the amino acid sequence of SEQ ID NO: 1.

3. The method according to claim 1, wherein the vesicle-forming protein comprises an amino acid sequence having at least 90% identity to an amino acid sequence selected from the group consisting of SEQ ID NOs: 2 to 6.

4. The method according to claim 1, wherein the cell is an in vitro cell, and the extracellular fluid is a culture supernatant.

5. The method according to claim 1, wherein the cell is an in vivo cell, and the extracellular fluid is a biological fluid.

6. An exosome-like vesicle comprising:
(a) a microRNA,
(b) a microRNA-binding protein comprising a first portion consisting of an MID domain and a PIWI domain of an Argonaute protein and a second portion consisting of a virus protein R, and
(c) a nanocage composed of a vesicle-forming protein comprising an amino acid sequence having at least 90% identity to an amino acid sequence selected from the group consisting of SEQ ID NOs: 2 to 6.

7. The exosome-like vesicle according to claim 6, further comprising (d) a membrane fusion protein.

8. The method according to claim 2, wherein the vesicle-forming protein comprises an amino acid sequence having at least 90% identity to an amino acid sequence selected from the group consisting of SEQ ID NOs: 2 to 6.

9. The method according to claim 2, wherein the cell is an in vitro cell, and the extracellular fluid is a culture supernatant.

10. The method according to claim 3, wherein the cell is an in vitro cell, and the extracellular fluid is a culture supernatant.

11. The method according to claim 2, wherein the cell is an in vivo cell, and the extracellular fluid is a biological fluid.

12. The method according to claim 3, wherein the cell is an in vivo cell, and the extracellular fluid is a biological fluid.
